(19) United States
(12) Patent Application Publication
(10) Pub. No.: US 2014/0243319 A1
(43) Pub. Date: Aug. 28, 2014

(54) TREATMENTS TARGETING GAMMA-SYNUCLEIN EXPRESSION LEVELS

(71) Applicant: Anita Sidhu, Falls Church, VA (US)

(72) Inventor: Anita Sidhu, Falls Church, VA (US)

(73) Assignee: GEORGETOWN UNIVERSITY, Washington, DC (US)

(21) Appl. No.: 14/005,753

(22) PCT Filed: Mar. 16, 2012
(86) PCT No.: PCT/US12/29413

§ 371 Date: May 1, 2014

Related U.S. Application Data
(60) Provisional application No. 61/454,150, filed on Mar. 18, 2011.

(51) Int. Cl. A61K 31/55; G01N 33/50; A61K 31/337

(52) U.S. Cl. CPC A61K 31/55; A61K 31/337; G01N 33/5044; G01N 2500/10
USPC 514/217; 514/449; 506/9; 435/6.1; 435/7.1

(57) ABSTRACT

A combination treatment taking advantage of the synergistic effect of inhibiting γ-synuclein expression and chemotherapeutic agents to provide improved treatment of cancers, preferably stage III/IV cancers. A method for identifying improved cancer treatments.

### GOVERNMENT RIGHTS

Inventions disclosed herein were made in part with the assistance of government funding under grant no: R01MH075020. The government may have certain rights in the inventions.

### FIELD

Compositions that modulate expression of γ-synuclein and methods of treatment comprising modulation of expression of γ-synuclein are described.

### SUMMARY

Gamma synuclein expression is observed in cancer cells that are resistant to chemotherapy, for example, cancers that are resistant to taxol treatment. Compounds in the family of tri-cyclic antidepressants (TCA), for example desipramine, can act to suppress γ-synuclein expression in cancer cell lines that otherwise show high levels of γ-synuclein expression. Treatment of cancer cells that are identified as expressing γ-synuclein with a compound that reduces γ-synuclein expression in combination with chemotherapeutic compounds can provide a synergistic cell killing effect that is greater than the effect provided by administering either compound by itself.

A combination treatment taking advantage of the synergistic effect of TCA's and chemotherapeutic agents to provide improved treatment of cancers, preferably stage III/IV cancers, can include determining that the cancer cells to be treated are expressing γ-Syn. Determination of γ-Syn expression can be by direct assay of a cell sample. For example, the presence of γ-Syn can be determined by Western blot, another obseravtion of antibody binding, or other biochemical assay. Alternatively, determination of γ-Syn expression can be accomplished by determining the presence in the cells of RNA encoding γ-Syn. Determination of γ-Syn can also be accomplished by observation of characteristics associated with γ-Syn expressing cancer cells, for example cell morphology, cytometric observations, staining, phenotypes associated with γ-Syn expression, resistance to treatment with taxanes.

A combination treatment taking advantage of the synergistic effect of TCA's and chemotherapeutic agents to provide improved treatment of cancers, can include co-administering, to a patient in need thereof, an effective amount of a chemotherapeutic agent, preferably a MT targeting agent, and most preferably a taxane or taxane derivative together with an effective amount of a γ-Syn expression inhibitor, preferably a TCA or derivative thereof such as DMI. In addition, or alternatively, the combination treatment can include administering a γ-Syn expression inhibitor, preferably a TCA or derivative thereof, such as DMI prior to administration of the chemotherapeutic agent, such that γ-Syn expression is inhibited at the time that the chemotherapeutic agent is administered. The administration of—Syn expression inhibitor and chemotherapeutic agents can be repeated in alternating or overlapping cycles to achieve a further effect.

A method for identifying improved methods of killing cancer cells that express γ-Syn can comprise determining the ability of an agent to inhibit γ-Syn expression in a γ-Syn expressing cancer cell and determining the ability of the γ-Syn expression inhibiting agent to enhance the cytotoxic effectiveness of a chemotherapeutic agent such as a taxane or taxane derivative in killing γ-Syn expressing cancer cells. An improved method of killing cancer cells can comprise co-administration of an agent effective to inhibit γ-Syn expression identified by such a method with a chemotherapeutic agent whose effectiveness is increased by inhibition of γ-Syn, or pretreatment with the agent effective to inhibit γ-Syn expression followed by administration or co-administration of an effective amount of the chemotherapeutic agent such that γ-Syn expression is inhibited at the time that the chemotherapeutic agent is administered.

### BRIEF DESCRIPTION OF THE DRAWING FIGURES

FIG. 1 shows improved behavior in WKY rats treated with antidepressants.

FIG. 2 shows that antidepressants reduce γ-Syn levels in the brain of WKY rats.

FIG. 3 shows—Syn protein levels are reduced in brain of WKY rats after desipramine treatment.

FIG. 4 shows Western blot data demonstrating γ-Syn expression in various cancer cell lines.

FIG. 5 shows that T47D breast cancer cells expressing γ-Syn are resistant to taxol.

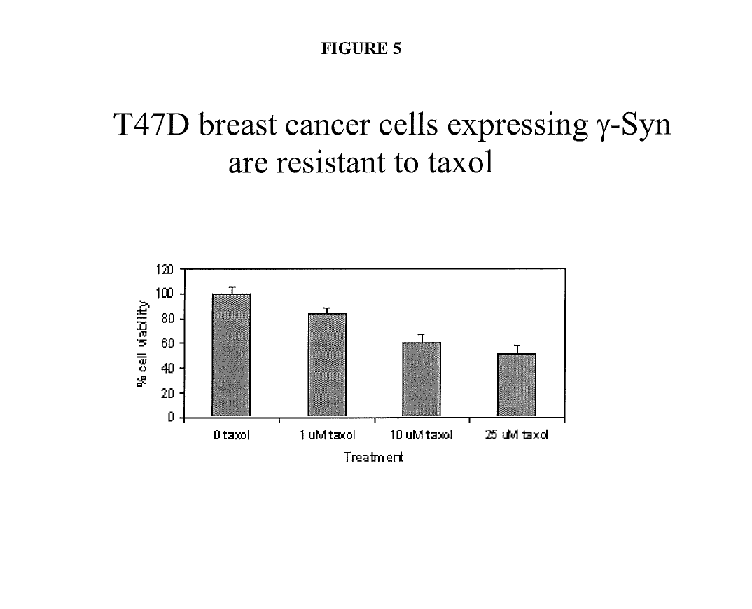

FIG. 6 shows that desipramine reduces γ-Syn levels in T47D breast cancer cells.

FIG. 7 shows results of a viability assay of T47D cells treated with desipramine and taxol.

FIG. 8 shows results of a viability assay of SK-BR-3 cells treated with desipramine and taxol.

FIG. 9 shows results of a viability assay of HT-29 cells treated with desipramine and taxol.

FIG. 10 shows results of a viability assay of HCT-116 cells treated with desipramine and taxol.

FIG. 11 shows results of a viability assay of A549 cells treated with desipramine and taxol.

FIG. 12 shows results of a viability assay of T47D cells treated with imipramine and taxol.

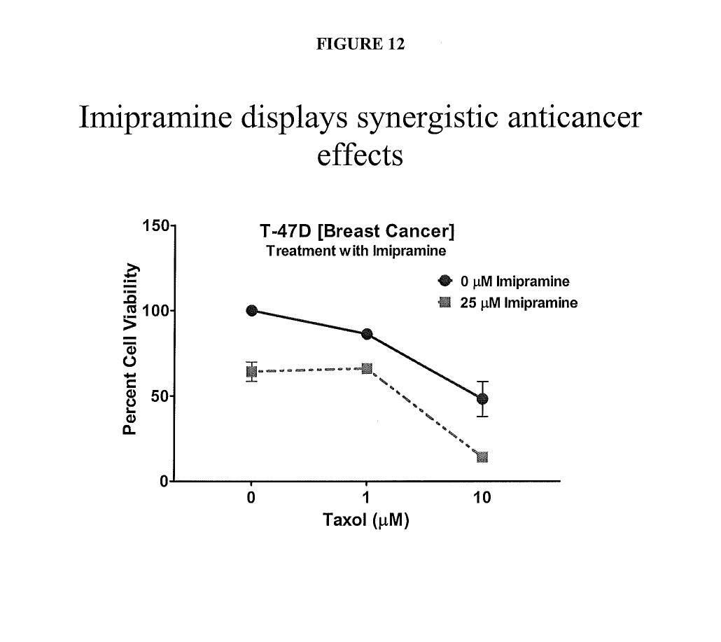

FIG. 13 shows Western blot data showing expression of γ-Syn pancreatic cancer cells

FIG. 14 shows results of a viability assay of COLO-357 cells treated with desipramine and taxol.

FIG. 15 shows results of a viability assay of BXPC-3 cells treated with desipramine and taxol.

FIG. 16 shows results of a viability assay of ASPC-1 cells treated with desipramine and taxol.

### DETAILED DESCRIPTION

The synucleins (α-, β- and γ-) are a family of small soluble proteins that are normally expressed in presynaptic neurons in the brain. α-synuclein (α-Syn) has been linked to the genesis of neurodegenerative disorders, such as Parkinson's disease. In a rat depression model, γ-synuclein (γ-Syn) is overexpressed in the brain. Treatment of the rats with the antidepressant desipramine (DMI) reduces expression levels of γ-Syn and relieves their depressive state (Jeannotte AM, McCarthy JG, Redei EE, Sidhu A (2009) “Desipramine Modulation of alpha-, gamma-Synuclein,and the Norepinephrine Transporter in an Animal Model of Depression” Neuropsychopharmacology 34(4):987-998).

γ-Syn is also known as breast carcinoma specific gene. It was initially isolated from infiltrating breast carcinoma cells. γ-Syn is not normally expressed in normal breast tissue, benign tumors or stage I/II cancers, and its expression in breast cancer (BC) is strongly associated with advanced stages of disease progression, where it has been found to be overexpressed in >70% of Stage III/IV breast and ovarian tumors. γ-Syn overexpression is also seen in a wide variety of other carcinomas such as colorectal, bladder, pancreatic, glaucoma, brain tumor and prostate cancer, where disease progression to stage III/IV is correlated with overexpression of this protein. In breast carcinoma and ovarian carcinoma, the aberrant expression of γ-Syn is thought to be promoted by hypomethylation of the CpG islands of the gamma-synuclein gene. Once overexpressed, gamma-synuclein is thought to promote cancer cell survival and inhibit stress- and chemotherapy drug-induced apoptosis by modulating MAPK pathways.

γ-Syn is an oncogene that is overexpressed in >70% of stage III/IV carcinomas but not in stage I or II cancers. Some of the cancers to which the overexpression of γ-Syn is linked to include breast, ovarian, colorectal, bladder, pancreatic, glaucoma, brain tumor and prostate cancer. Patients with γ-Syn positive cancers have a significantly shorter disease-free survival and overall survival, compared to patients that do not express γ-Syn.

Microtubule (MT) targeting agents, such as taxanes, are currently the first line of chemotherapeutic agents to treat patients with advanced or metastatic cancer, but stage III/IV patient response to taxanes varies significantly. These MT targeting agents rely heavily on the normal function of the mitotic checkpoint machinery, including BubR1, a mitotic checkpoint kinase whose activity is inhibited by γ-Syn. The interaction of γ-Syn with BubR1 results in impairment of the mitotic checkpoint machinery, rendering these cells resistant to MT destabilizers.

Expression of γ-Syn results in increased resistance of these cells to MT targeting agents. Moreover, in vitro studies pretreating cells with either the cytokine oncostatin M or injecting cells individually with a small anti-γ-Syn peptide have shown that inhibition of γ-Syn expression increases cell susceptibility to MT destabilizing agents. However, none of these methods of decreasing γ-Syn levels are suitable for treatment of patients.

γ-Syn promotes cell survival and proliferation, inhibits stress and current MT inhibitory drug-induced apoptosis, activates estrogen receptor, inhibits mitotic checkpoint control and promotes metastasis in a nude mouse model. Stage III/IV cancers are more resistant to current MT targeting chemotherapies, such as taxol, and in breast cancer cells overexpressing γ-Syn, the presence of γ-Syn renders these cells resistant to these agents. Conversely, decreasing the expression of γ-Syn in cells, through an anti-γ-Syn peptide injected into cells, or upon pretreatment of cells with the cytokine oncostatin M, renders these cells much more susceptible to taxol. Thus, treatment strategies that reduce γ-Syn expression levels can provide improved treatment of stage III/IV cancers.

Pancreatic ductal adenocarcinoma (PC) is currently the fourth leading cause of cancer-related death in Western countries. Long-term survival is rare, with the overall 5-year survival rates ranging from 10% to 25%. In normal pancreas, islet cells and some acinar cells express low levels of γ-Syn but the ductal epithelium is negative for γ-Syn. Very little is known about the role of γ-Syn in PC. In one study, 22 of 32 pancreatic tumor tissue samples (69%) were found to express γ-Syn. Unlike previous findings with breast cancer where γ-Syn expression was found only in stage III/IV cancers but not in stage I/II cancers, γ-Syn was shown to be present in 61% of the tumor tissue samples examined from patients with Stage I and II pancreatic carcinoma. The overexpression of γ-Syn was correlated with perineural and lymph node invasion. This finding was subsequently also confirmed by another study on γ-Syn in pancreatic cancer. Through proteomics and transcriptomics, γ-Syn was found to be the only protein that was up-regulated in high perineural invasive PC, confirming a role for this protein in pancreatic carcinoma invasion. Multivariate analyses revealed γ-Syn overexpression as the only independent predictor of diminished overall survival and the strongest negative indicator of disease-free survival associated with PC. γ-Syn was detected in serum samples from 21 of 56 patients (38%) with pancreatic carcinoma, suggesting that this protein can be a useful biomarker.

DMI, an FDA-approved tricyclic antidepressant, can reduce γ-Syn expression in PC cells at clinically relevant concentrations and cause cell death (>60%). Even low concentrations of the antidepressant, when given in combination with taxol, results in >60-80% cell death of PC cells. Furthermore, overexpression of γ-Syn is also present in a large variety of other stage III/IV cancers, for example, breast, ovarian, colorectal, bladder, pancreatic, glaucoma, brain tumor and prostate cancer. Consequently, DMI treatment, or DMI pretreatment, followed by taxol, can provide improvements in the treatment of other advanced stage III/IV metastatic cancers as well. Since the treatment can use FDA-approved drugs, and is used at clinically relevant doses, this treatment can be immediately used. More generally, these findings demonstrate that treatment of advanced stage III/IV metastatic cancers.

γ-Syn was found to be the only protein overexpressed in high perineural invasive PCs, an especially aggressive form of this cancer. Multivariate analyses revealed γ-Syn overexpression as the only independent predictor of diminished overall survival and the strongest negative indicator of disease-free survival associated with PC.

In brain, γ-Syn is expressed in presynaptic terminals of monoaminergic neurons, and our lab has shown that this protein can regulate the function of norepinephrine and serotonin transporters. Unlike α-Syn, whose role in neurodegenerative diseases such as Parkinson's disease is well established, a role for γ-Syn in neurodegeneration is not known. γ-Syn is overexpressed in the frontal cortex of the WKY, a rat model of depression. Chronic treatment of the WKY rat with DMI for 2 weeks, caused a decrease in γ-Syn levels in frontal cortex of brain. This finding was unexpected since DMI is a known inhibitor of norepinephrine transporter (NET), which acts to block its norepinephrine reuptake activity. The reduction in γ-Syn levels was accompanied by a relief in the depressive symptoms, and led to restoration of normal NET activity, including sensitivity of NET to the chemotherapeutic/MT destabilizing agent, nocodazole. This latter finding showed that γ-Syn binds tightly to MTs, preventing normal cytosolic-cell surface trafficking of the norepinephrine transporter. The ability of γ-Syn to bind tightly to MTs may be one mechanism by which its overexpression in carcinomas protects cells from the MT actions of chemotherapeutic agents, for example, taxol, nocodazole, vinblastine and colchicine.

The ability of DMI to reduce γ-Syn in the WKY rat was unexpected. DMI is a tricyclic antidepressant. Tricyclic antidepressants (TCAs) are a class of psychoactive drugs used primarily as antidepressants. They are named after their chemical structure, which contains three rings of atoms. TCAs include the following: tertiary amines, e.g., Amitriptyline (Elavil), Amitriptylinoxide (Ambivalon, Equilibrin), Butriptyline (Evadyne), Clomipramine (Anafranil); Dosulepin/Dothiepin (Prothiaden), Doxepin (Adapin, Sinequan), Imipramine (Tofranil), Imipraminoxide (Imiprex, Elepsin), Lofepramine (Lomont, Gamanil), Trimipramine (Surmontil), and secondary amines, e.g., Desipramine (Norpramin, Pertofrane); Nortriptyline (Pamelor, Aventyl); Protriptyline (Vivactil); and also includes: Demexiptiline (Deparon, Tinoran), Dibenzepin (Noveril, Victoril), Dimetacrine (Istonil, Istonyl, Miroistonil), Iprindole (Prondol), Melitracen (Deanxit, Dixeran, Melixeran, Trausabun), Metapramine (Timaxel), Nitroxazepine (Sintamil), Noxiptiline (Nogedal), Propizepine (Vagran), Quinupramine (Kevopril, Kinupril, Adeprim, Quinuprine), Amineptine (Survector, Maneon, Directim), Opipramol (Insidon, Pramolan, Ensidon, Oprimol), Tianeptine (Stablon, Coaxil, Tatinol), Cianopramine (Ro-11-2465), Cyanodothiepin (BTS-56,424), and Fluotracen (SKF-28,175).

TCAs are generally understood to act primarily as serotonin-norepinephrine reuptake inhibitors (SNRIs) by blocking the serotonin transporter (SERT) and the norepinephrine transporter (NET), respectively, which results in an elevation of the extracellular concentrations of these neurotransmitters, and therefore an enhancement of neurotransmission. In addition to their reuptake inhibition, many TCAs also have high affinity as antagonists at the 5-HT2 (5-HT2A and 5-HT2C), 5-HT6, 5-HT7, al-adrenergic, and NMDA receptors, and as agonists at the sigma receptors (σ1 and σ2), some of which may contribute to their therapeutic efficacy, as well as their side effects. The TCAs also have varying but typically high affinity for antagonizing the H1 and H2 histamine receptors, as well as the muscarinic acetylcholine receptors. As a result, they also act as potent antihistamines and anticholinergics. Most, if not all, of the TCAs also potently inhibit sodium channels and L-type calcium channels, and therefore act as sodium channel blockers and calcium channel blockers, respectively.

The results described above reveal another function of molecules in the tri-cyclic antidepressant family and derivatives thereof. Through inhibition of γ-Syn expression, these compounds can be used to synergistically improve the effect of chemotherapeutic agents. A preferred class of chemotherapeutic agents for use in combination with TCA's are the MT targeting compounds, for example taxanes and derivatives thereof. The taxanes are diterpenes produced by the plants of the genus Taxus (yews). As their name suggests, they were first derived from natural sources, but some have been synthesized artificially. Taxanes include Docetaxel, Larotaxel, Ortataxel, Paclitaxel (Taxol), Tesetaxel, and Epothilones (Ixabepilone).

A combination treatment taking advantage of the synergistic effect of TCA's and chemotherapeutic agents to provide improved treatment of cancers, preferably stage III/IV cancers, can include determining that the cancer cells to be treated are expressing γ-Syn. Determination of γ-Syn expression can be by direct assay of a cell sample. For example, the presence of γ-Syn can be determined by Western blot, another obseravtion of antibody binding, or other biochemical assay. Alternatively, determination of γ-Syn expression can be accomplished by determining the presence in the cells of RNA encoding γ-Syn. Determination of γ-Syn can also be accomplished by observation of characteristics associated with γ-Syn expressing cancer cells, for example cell morphology, cytometric observations, staining, phenotypes associated with γ-Syn expression, resistance to treatment with taxanes.

A combination treatment taking advantage of the synergistic effect of TCA's and chemotherapeutic agents to provide improved treatment of cancers, can include co-administering, to a patient in need thereof, an effective amount of a chemotherapeutic agent, preferably a MT targeting agent, and most preferably a taxane or taxane derivative together with a γ-Syn expression inhibitor, preferably a TCA or derivative thereof such as DMI. In addition, or alternatively, the combination treatment can include administering an effective amount of a γ-Syn expression inhibitor, preferably a TCA or derivative thereof, such as DMI prior to administration of an effective amount of the chemotherapeutic agent, such that γ-Syn expression is inhibited at the time that the chemotherapeutic agent is administered. The administration of—Syn expression inhibitor and chemotherapeutic agents can be repeated in alternating or overlapping cycles to achieve a further effect.

Because of the synergistic effect provided by TCA's such as desipramine it is possible to use lower dosages of chemotherapeutic agents when used in combination with a TCA such as DMI. Accordingly, the methods of treatment described above can also permit using lower than the FDA recommended dosages of the chemotherapeutic agent.

A method for identifying improved methods of killing cancer cells that express γ-Syn can comprise determining the ability of an agent to inhibit γ-Syn expression in γ-Syn expressing cancer cells and determining the ability of the γ-Syn expression inhibiting agent to enhance the cytotoxic effectiveness of a chemotherapeutic agent, for example a MT targeting chemotherapeutic agent, and/or a mitosis checkpoint targeting agent, for example a taxane or taxane derivative, in killing the γ-Syn expressing cancer cells. An improved method of killing cancer cells can comprise co-administration of an agent effective to inhibit γ-Syn expression identified by such a method with a chemotherapeutic agent whose effectiveness is increased by inhibition of γ-Syn, or pretreatment with the agent effective to inhibit γ-Syn expression followed by administration or co-administration of the chemotherapeutic agent such that γ-Syn expression is inhibited at the time that the chemotherapeutic agent is administered.

### EXAMPLES

### Example 1

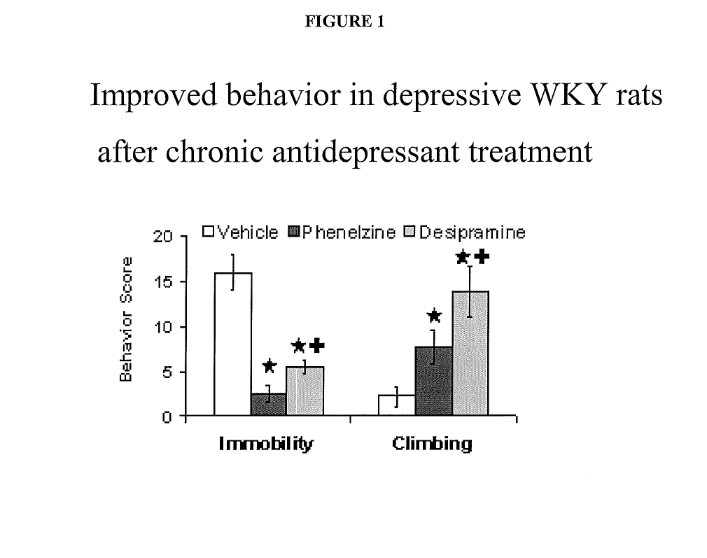

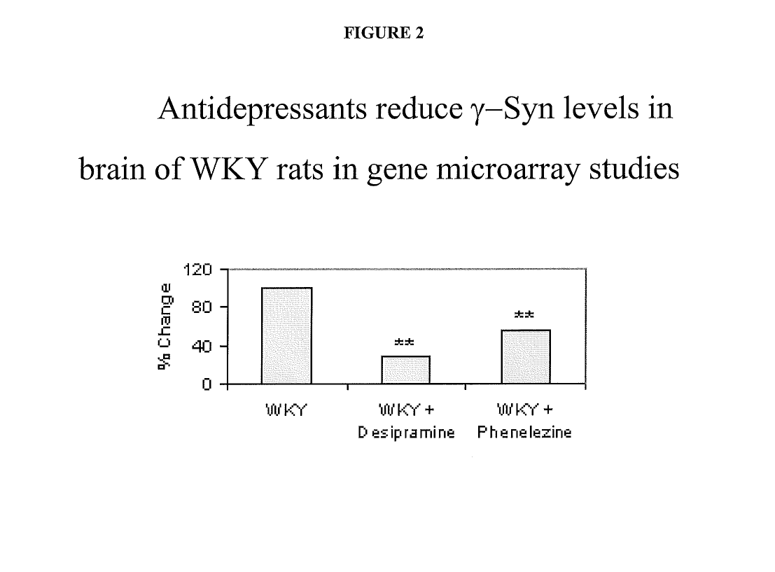

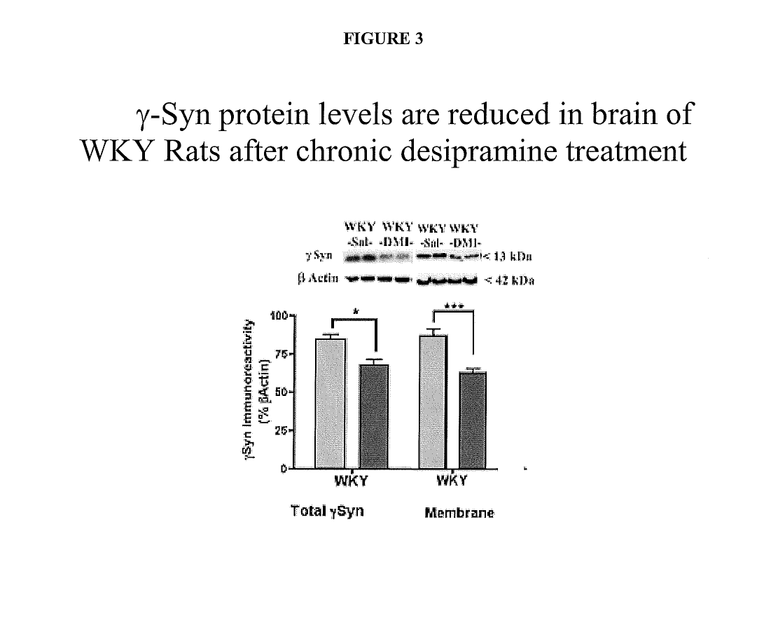

The effect of the TCA compound DMI was tested in a depression model using WKY rats. Rats receiving chronic administration of DMI and phenelezine showed improvements in mobility and climbing behaviors (FIG. 1). Surprisingly, a gene expression microarray study showed that the antidepressants reduced γ-Syn expression levels (FIG. 2). Western blotting studies also demonstrated reduced levels of γ-Syn protein in the brains of DMI treated WKY rats (FIG. 3).

### Example 2

Cancer cell lines were tested to assess if inhibitors of γ-Syn expression could be an effective treatment in reducing γ-Syn levels in cancer cells, and if reducing γ-Syn expression also resulted in diminishing their viability.

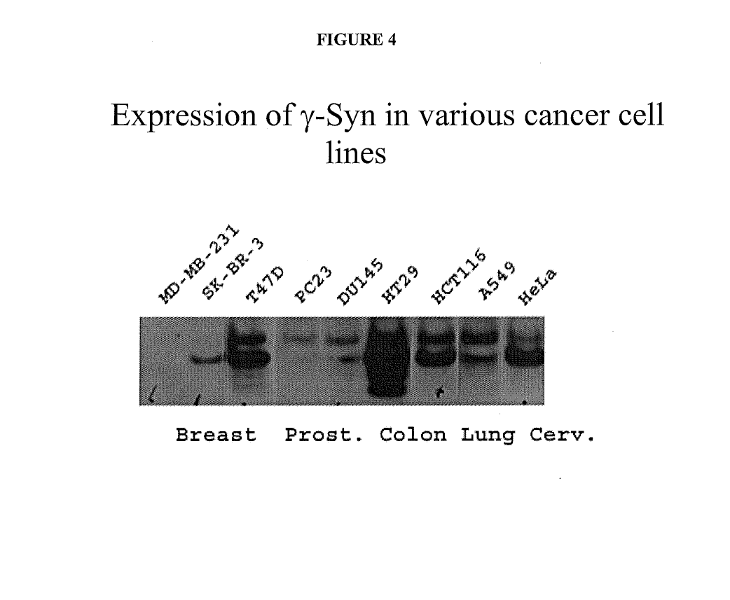

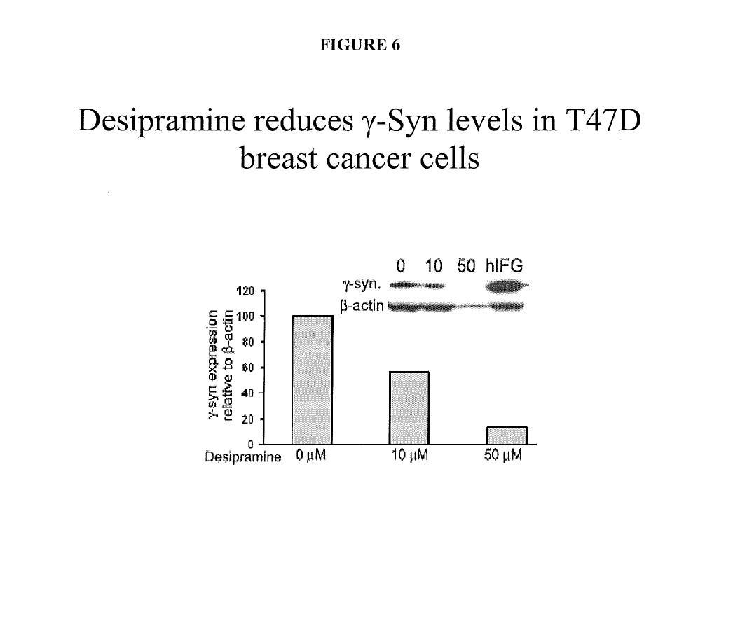

Various cancer cell lines were tested for expression of γ-Syn by Western blots. (FIG. 4) Two breast carcinoma cell lines were tested for γ-Syn expression by Western blots, and human brain (hIFG) was used as a positive control. T47D cells expressed high levels of γ-Syn (FIG. 6), while SK-BR-3 cells had low γ-Syn levels. Pretreatment of T47D cells for 48 h with 10 or 50 μM DMI resulted in a decrease of γ-Syn levels, by 40 and 85%, respectively. (FIG. 6) These results show that DMI can reduce the protein levels of γ-Syn in endogenously expressing BC cells. Moreover, such reduction occurred in the absence of the norepinephrine transporter, suggesting that DMI has a direct effect on γ-Syn independent of the transporter.

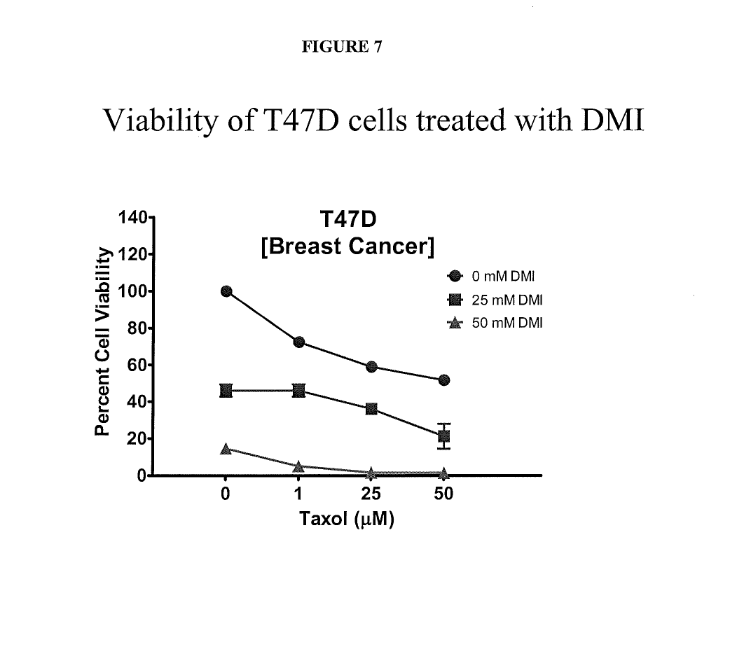

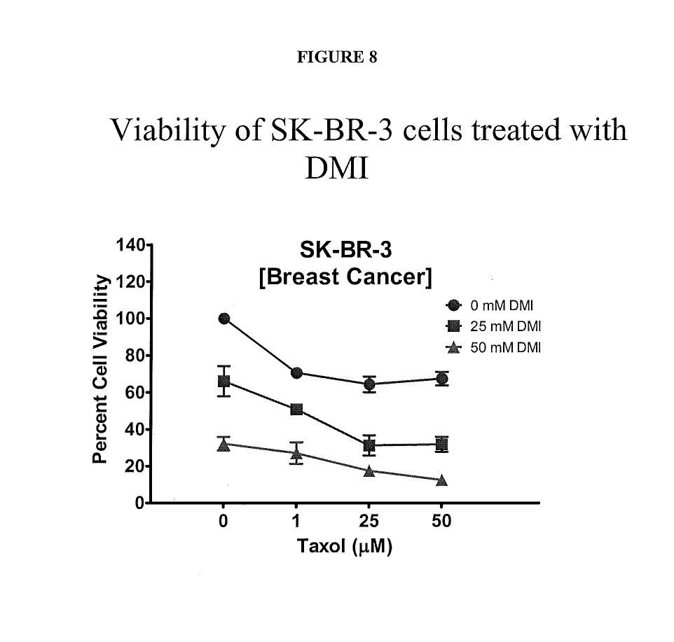

DMI+taxol reduces T47D cell viability. (FIG. 7) T47D cells were treated with varying levels of taxol or DMI for 48 h and cell viability was measured through MTT assays. In the presence of taxol alone, cell viability was high (52% at 25 μM taxol), consistent with resistance of γ-Syn-expressing cells to taxol. Cells treated with DMI alone also were similarly resistant to DMI (58% cell viability at 25 μM DMI). However, at 50 μM DMI, cell viability was only ˜5% (data not shown) suggesting that DMI alone at high concentrations can be useful in inducing cell death. DMI+taxol reduces SK-BR-3 cell viability. (FIG. 8)

Cells were next pretreated with DMI for 24h, followed by an additional 24 h with DMI+taxol. Cell viability was dramatically decreased. At 10 μM of either DMI or taxol, cell viability was ˜80%. In the presence of both DMI+taxol (10 μM each), cell viability was sharply reduced to 25%. A similar pattern was also seen at other combinations of both drugs, suggesting that first reducing γ-Syn levels with DMI, markedly increases cell susceptibility to taxol.

### Example 3

Several breast cancer T47D cells are observed to express high levels of γ-Syn (FIG. 4) and are resistant to taxol treatment. Desipramine treatment at 10 μM and 50 μM reduces γ-Syn levels.

Cells were selected that expressed γ-Syn. The selected cells were treated with varying concentrations of DMI (0-50 μM) for 48 h. The treatments resulted in a dose-dependent decrease in γ-Syn protein expression; at 50 μM DMI, >85% of γ-Syn protein expression was decreased. We next measured cell death by MTT assays and found a direct correlation between loss of γ-Syn protein expression and cell death. Only cells that expressed γ-Syn were susceptible to 50 μM DMI, with >90% cell death; cells that did not express any γ-Syn were completely resistant to DMI, with no cell death.

If cells were pretreated for 24 h with lower levels of DMI (10-25 μM), followed by 24 h treatment with varying concentrations of taxol, there was a synergistic effect of the two compounds. In other words, each compound was more effective when used together (˜60% cell death), than when used alone (˜15% cell death for each compound). This synergy permitted both these compounds to be used at clinically relevant doses (-10-15 μM each). These findings demonstrate that this strategy is useful for the clinical treatment of stage III/IV cancers in cells expressing γ-Syn. In the absence of γ-Syn expression, the synergistic effect of the two compounds was lacking. Therefore, it is generally preferable to determine the existence of γ-Syn expression by the cancer cells of a patient prior to initiating a combination therapy. IC50 values for treatment with DMI alone and in combination with taxol demonstrate the synergistic effect in γ-Syn expressing cell lines.

### Example 4

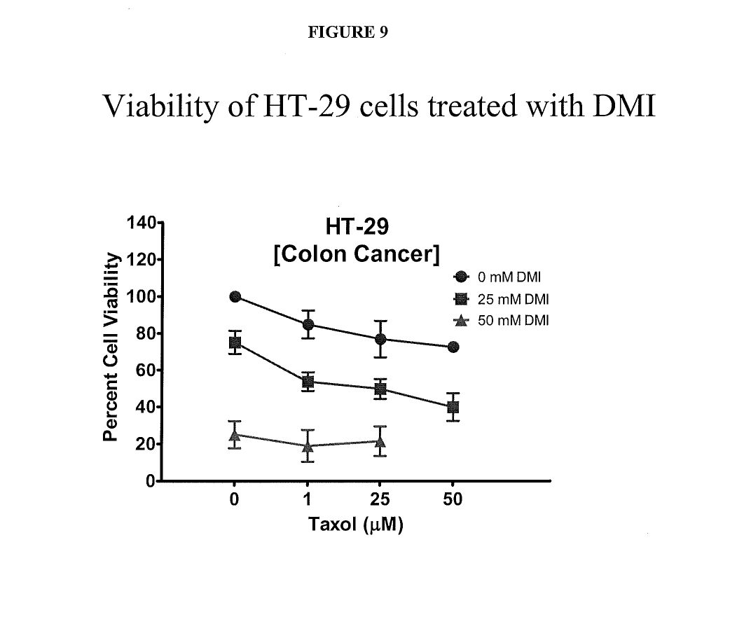

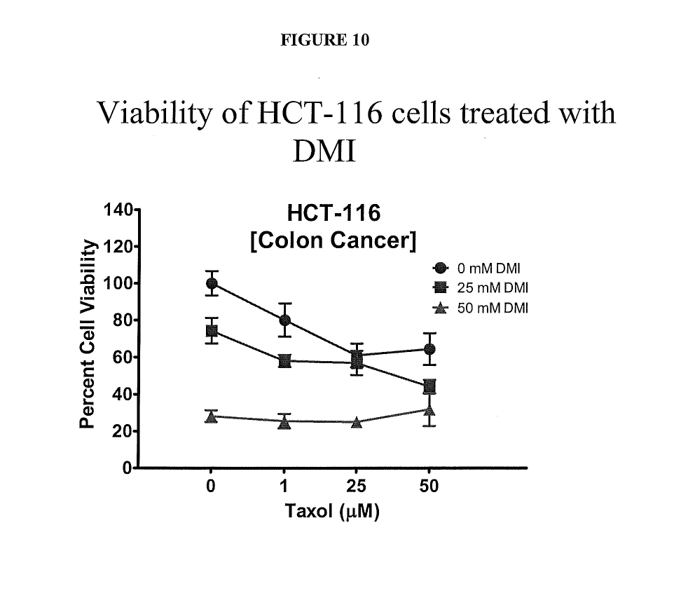

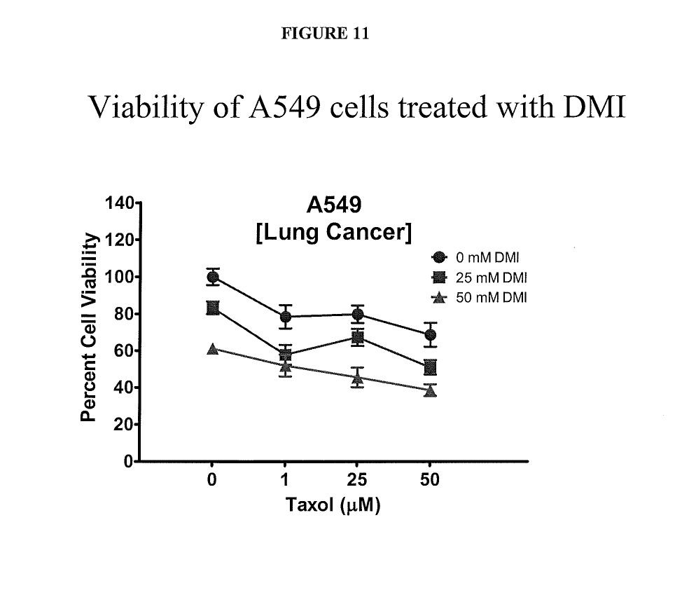

Cell viability assays were conducted as described above for a variety of cancer cell lines that express γ-Syn. (FIG. 4). Treatment with desipramine and taxol showed a synergistic effect of the combined treatments. FIGS. 6-11. Imipramine also demonstrated a synergistic effect in combination with taxol on cytotoxicity of T47D breast cancer cells.

### Example 5

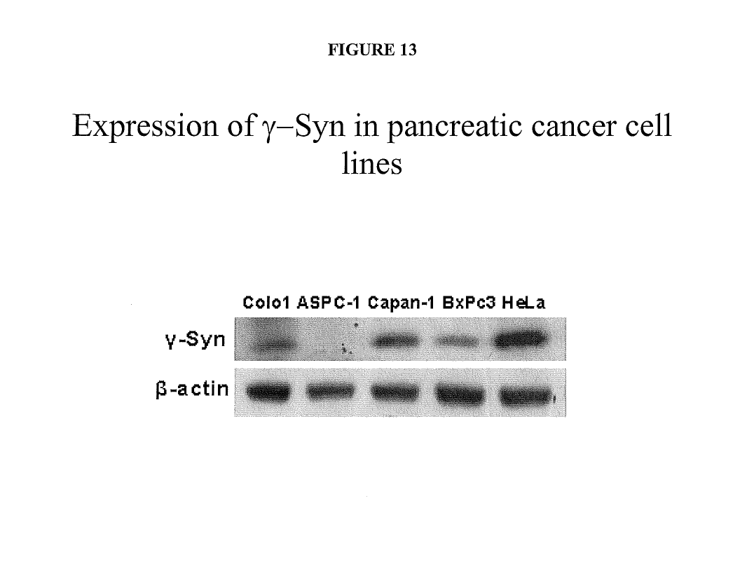

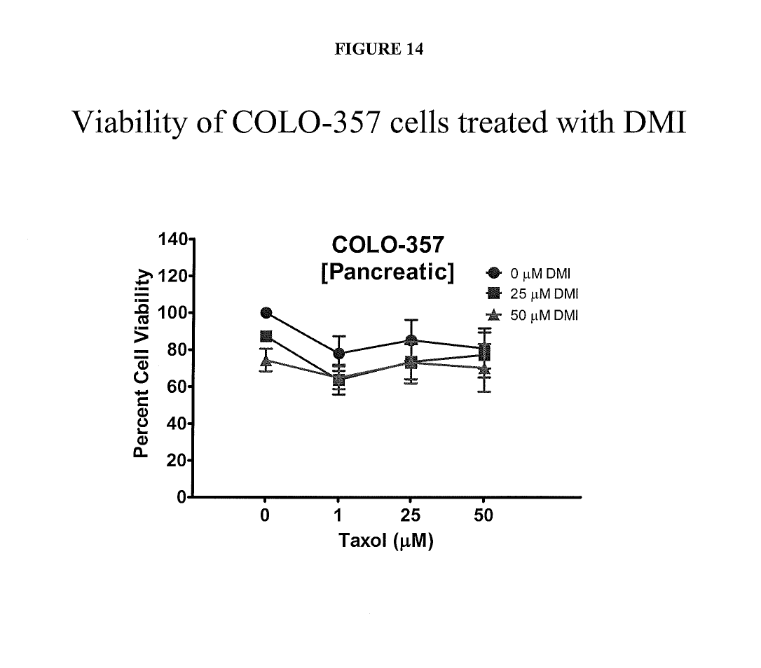

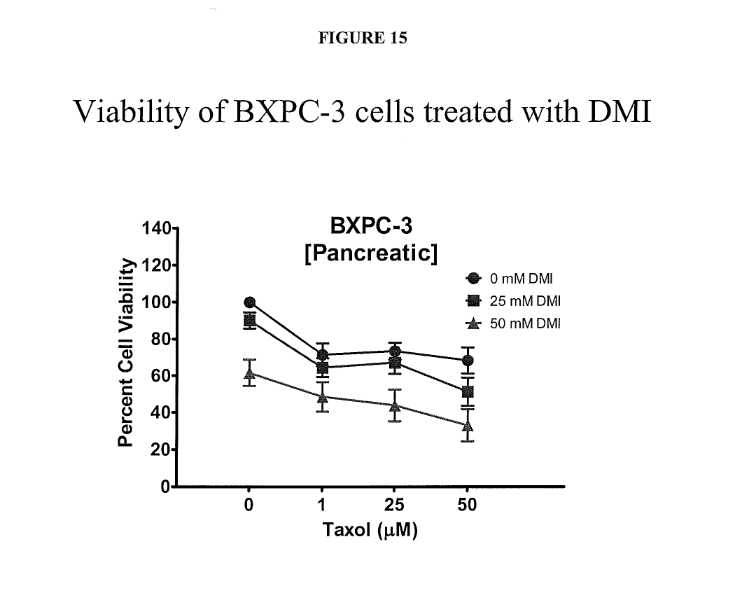

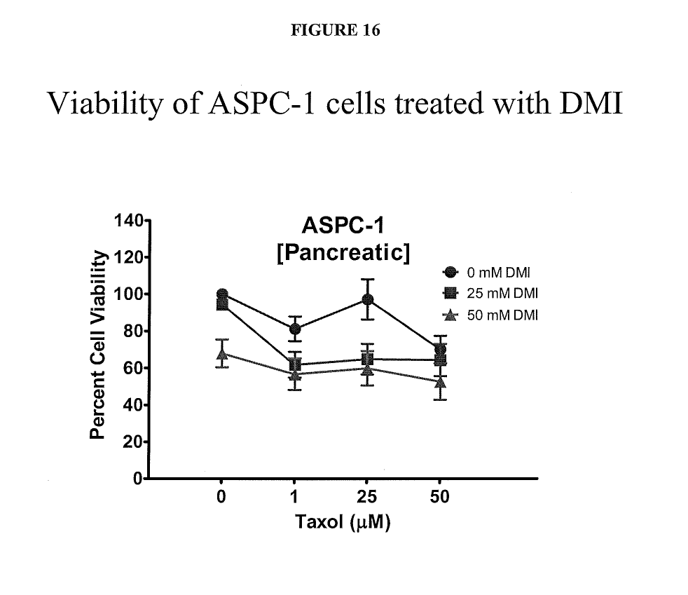

Various pancreatic cancer cell lines were assayed for γ-Syn expression. (FIG. 13). Cell lines which showed γ-Syn expression also showed a synergistic cytotoxicity effect of combined treatment of the cells with desipramine and taxol in assays conducted as described above. (FIGS. 14-16).

TABLE I

IC50 values of
cell death in μM

| Cell line | DMI + taxol | DMI |
|---|---|---|
| HCT 116 | 27.7 ± 5.5 | 43 ± 3 |
| HT-29 | 15.5 ± 4 | 38 |
| A549 | 11.3 ± 6.4 | >50 |
| DU145 | 4 ± 3 | >50 |
| T47D | 18 | 35 |
| HeLa | 37 | 43 |
| SK-BR-3 | 10 | 40 |
| MD-MB-231 | >50 | >50 |
| ASPC-1 | >50 | >50 |
| Colo-357 | >50 | >50 |
| BxPC-3 | 1 | >50 |

While exemplary articles and methods have been described in detail with reference to specific embodiments thereof, it will be apparent to those skilled in the art that various changes and modifications can be made, and equivalents employed without departing from the scope of the pending claims.

Each publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the, scope of the present invention be limited solely by the scope of the following claims, including equivalents thereof.

1. A method for the treatment of cancers involving cancer cells that express γ-Syn, the method comprising:
determining that cancer cells of the cancer to be treated in a patient in need of treatment are expressing γ-Syn,
administering an effective dosage regimen of an agent effective to suppress γ-Syn expression in the cancer cells, and
administering an effective dosage regimen of a chemotherapeutic agent such that γ-Syn expression is inhibited in the cancer cells at the time that the chemotherapeutic agent is administered.

2. The method of claim 1, wherein determining that cancer cells of the cancer to be treated in the patient in need of treatment are expressing γ-Syn comprises performing an assay for the presence of the γ-Syn protein in a sample of the cancer cells.

3. The method of claim 1, wherein determining that cancer cells of the cancer to be treated in the patient in need of treatment are expressing γ-Syn comprises performing an assay for the presence of RNA encoding the γ-Syn protein in a sample of the cancer cells.

4. The method of claim 1, wherein determining that cancer cells of the cancer to be treated in the patient in need of treatment are expressing γ-Syn comprises performing an assay for the comprises observation of characteristics of the cancer cells indicating the presence of γ-Syn expression in the cancer cells.

5. The method of claim 1, wherein administering an effective dosage regimen of an agent effective to suppress γ-Syn expression in the cancer cells comprises administering an effective amount of a γ-Syn expression inhibition agent chronically throughout a treatment period.

6. The method of claim 1, wherein administering an effective dosage regimen of an agent effective to suppress γ-Syn expression in the cancer cells comprises administering an effective amount of a γ-Syn expression inhibition agent prior to administration of a chemotherapeutic agent.

7. The method of claim 1, wherein administering an effective dosage regimen of an agent effective to suppress γ-Syn expression in the cancer cells comprises administering an effective amount of a γ-Syn expression inhibition agent prior to and together with administration of a chemotherapeutic agent.

8. The method of claim 1, wherein administering an effective dosage regimen of an agent effective to suppress γ-Syn expression in the cancer cells comprises administering a tricyclic antidepressant.

9. The method of claim 1, wherein administering an effective dosage regimen of an agent effective to suppress γ-Syn expression in the cancer cells comprises administering desipramine.

10. The method of claim 1, wherein administering an effective dosage regimen of an agent effective to suppress γ-Syn expression in the cancer cells comprises administering imipramine.

11. The method of claim 1 wherein administering an effective dosage regimen of a chemotherapeutic agent comprises administering a taxane.

12. The method of claim 1 wherein administering an effective dosage regimen of a chemotherapeutic agent comprises administering taxol.

13. A method for identifying improved methods of killing cancer cells that express γ-Syn, the method comprising
determining the ability of an agent to inhibit γ-Syn expression in γ-Syn expressing cancer cells, and
determining the ability of the γ-Syn expression inhibiting agent to enhance the cytotoxic effectiveness of a chemotherapeutic agent.

14. An improved method of killing cancer cells that express γ-Syn, the method comprising,
administering to an individual subject in need thereof an effective amount of an agent effective to inhibit γ-Syn expression that has been identified by the method of claim 12 and
administering an effective amount of the chemotherapeutic agent whose effectiveness is increased by inhibition of γ-Syn,
wherein administering the agent effective to inhibit γ-Syn expression comprises co-administration with the chemotherapeutic agent and/or pretreatment with the agent effective to inhibit γ-Syn expression followed by administration or co-administration of the chemotherapeutic agent such that γ-Syn expression is inhibited at the time that the chemotherapeutic agent is administered.

15. A method of killing cancer cells that express γ-Syn, the method comprising:
determining that cancer cells are expressing γ-Syn,
exposing the cancer cells to an effective dosage regimen of an agent effective to suppress γ-Syn expression in the cancer cells, and
exposing the cancer cells to an effective dosage regimen of a
chemotherapeutic agent such that γ-Syn expression is inhibited in the cancer cells at the time that the chemotherapeutic agent is administered.

16. The method of claim 14, wherein determining that the cancer cells are expressing γ-Syn comprises performing an assay for the presence of the γ-Syn protein or RNA encoding the γ-Syn protein in a sample of the cancer cells.

17. The method of claim 14, comprising exposing the cancer cells to an effective amount of a γ-Syn expression inhibition agent prior to exposing the cancer cells to a chemotherapeutic agent.

18. The method of claim 14, wherein exposing the cancer cells to effective dosage regimen of an agent effective to suppress γ-Syn expression in the cancer cells comprises exposing the cancer cells to a tricyclic antidepressant.

19. The method of claim 14, wherein exposing the cancer cells to an effective dosage regimen of an agent effective to suppress γ-Syn expression in the cancer cells comprises exposing the cancer cells to desipramine.

20. The method of claim 14, wherein exposing the cancer cells to an effective dosage regimen of an agent effective to suppress γ-Syn expression in the cancer cells comprises exposing the cancer cells to imipramine.

21. The method of claim 14, wherein exposing the cancer cells to an effective dosage regimen of a chemo therapeutic agent comprises exposing the cancer cells to a taxane.
